(19) United States
(12) Patent Application Publication
(10) Pub. No.: US 2006/0029659 A1
(43) Pub. Date: Feb. 9, 2006

(54) Immune dengue plus

(76) Inventor: Michael Panzardi, San Juan, PR (US)

Correspondence Address: DR. MICHAEL PANZARDI, P.O. BOX 6464, SAN JUAN, PR, 00914, US

(21) Appl. No.: 10/912,987

(22) Filed: Aug. 6, 2004

(51) Int. Cl. A61K 36/38; A61K 36/28; A61K 9/48; A61K 35/64

(52) U.S. Cl. 424/451; 424/730; 424/737; 424/539

(57) ABSTRACT

An immune enhancing blood platelet builder that increases the survival rate of the patient against the duration of the dengue fever virus in the body.

### BRIEF SUMMARY OF THE INVENTION

This invention is an immune enhancing blood platelet builder that increases the survival rate of the patient against the duration of the dengue fever virus in the body.

### BACKGROUND OF THE INVENTION

Upon visiting the local industrial promotion governmental agency, Fomento, the conversation was directed toward an observation made by the officers who commented their concern for dengue fever and the alarming rate of death caused by this virus. And they added that there should be some work done on this problem to produce a formula to combat this. It is not only a health hazard at any age, but it is also devastating to the tourism economy.

Dengue fever is carried by a particular species of mosquito called aeries egypti which has distinctive black and white markings on the appendages. It has the typical characteristic effect on the human body that upon stinging the reaction is a chemical ability to lower the number of blood platelets. Apart from causing the other asymptomatic physical problems of fever, extreme pain to joint areas, general weakness and discomfort, what sets it apart from other like-virus symptoms caused by other viruses, dengue fever virus causes a very localized distinct pain behind the orbit of the eye. This sets it apart from other common viruses in our ability to distinguish that it is dengue fever other than clinically proven rapid blood platelet reductions which eventually causes life-threatening internal hemorrhaging.

It so happened that the inventor (“he”) had already contracted the dengue fever virus that same year it was announced by the Public Health Department, and had developed the classic symptoms. He decided to confirm and identify the virus as such by asking the aid and cooperation of a clinical laboratory technician. Upon confirming through blood tests that they were dealing with dengue fever virus, the inventor decided to attempt to experiment on himself in order to possibly aid himself in correcting and/or offsetting the effects of the virus. The clinical technician was instructed by the inventor to take samples of his blood every two to three hours. Since the underlining dangers were the rapid fall of blood platelets, the inventor then focused his attention on trying certain herbs on himself that had the potential of raising the number of these platelets. In the event that was not discovered soon enough or didn't happen, the inventor proceeded also to administer immune-favorable aids or enhancers in an attempt to outlive the virus' survival time. During this process, a formula was discovered that upon measuring the clinical blood test effects, if any, it had on the blood, specifically looking for blood platelet increase or decrease, the inventor found that as he took this combination above, platelets increased eight to ten thousand per every eight to ten hours, taking the formula every two to four hours. The blood tests were conducted for a total of two days or 48 hours with an actual clinical blood test monitoring in any given day two hours for a total of ten hours of observation. There was an extraction of blood every two to three hours for the duration of time mentioned.

### SUMMARY OF THE INVENTION

This invention is a blood platelet builder that increases the number of blood platelets when in the presence of dengue fever. Referring to FIG. 1, this graph represents those groups of people who are in the 200,000 blood platelet range (A) who would be more at risk. During the first day, the virus reduces this number by about 30,000 platelets to level (B). During the second day, the number of platelets decreases by 40,000 to level (C). At level (D) on the third day, the patient very probably will start to hemorrhage and if not corrected, will eventually reach such low platelet counts that the loss of blood will cause their death. These results may vary from individual to individual depending on other general constitutional variables such as age and immune function providing there are no other physiological complications to start with.

Referring to FIG. 2, with the assistance of the formula, at the fourth day the graph shows that the patient is still above the hemorrhagic level (E). Considering that the virus usually only has a four day crisis duration, the patient is out of danger from hemorrhaging effects of the virus.

### A BRIEF DESCRIPTION OF THE DRAWINGS

FIG. 1. Graph showing decrease in blood platelets with dengue fever.

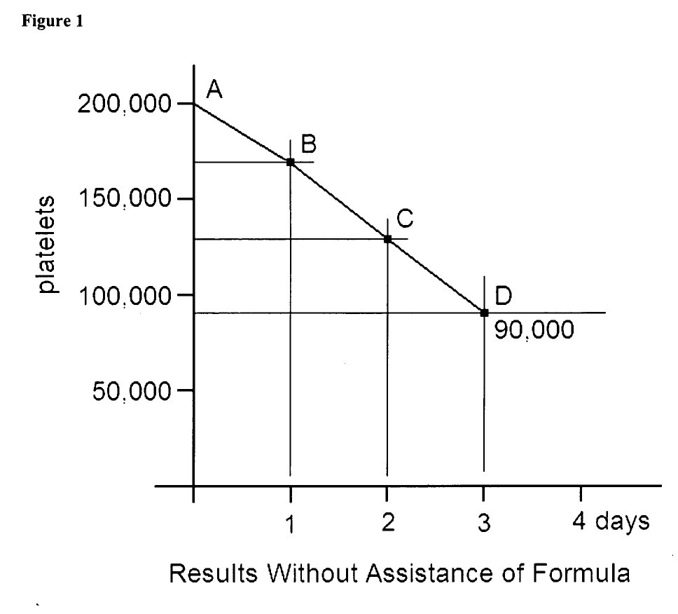

FIG. 2. Graph showing increase in blood platelets using formula.

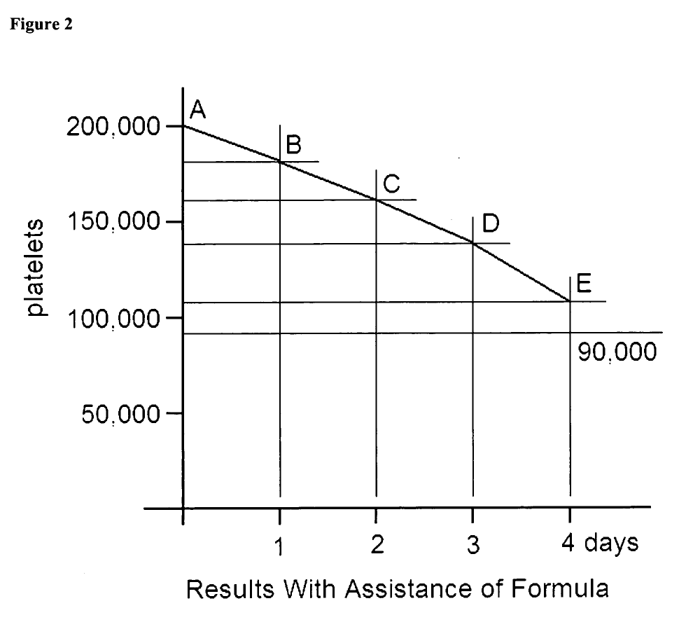

### DETAILED DESCRIPTION OF THE INVENTION

1. The herbs and nutrients that must be combined to reach these beneficial results are the following:
a. St.John's Wort
b. Echinacea
c. Bee propilus
d. Vitamin C

2. The dosage amount in this particular study for each of the following is:
a. 400 mg every 2 to 3 hours for St.John's Wort in a period of 10 to 12 hours or more.
b. 280 mg every 4 hours for Echinacea in a period of 10 to 12 hours.
c. 180 to 250 mg every 4 hours for Bee propilus in a period of 10 to 12 hours.
d. 500 to 1000 mg every 1 to 2 hours for Vitamin C in a period of 10 to 12 hours. For people who experience low tolerance like diarrhea or gastritis, they should consider a buffered Vitamin C.
e. Note of consideration: patient should be advised or mindful of taking the appropriate amount of liquids, such as 10 to 12 glasses of 8 ounces each or more of water everyday, in order to properly process the formula and to ensure a more speedy recovery.

Although the invention has been described with reference to specific embodiments, those skilled in the art will appreciate that many modifications and variations are possible without departing from the teachings of the invention. All such modifications and variations are intended to be encompassed within the scope of the following claims.

I claim:

1. An immune enhancing blood platelet builder formula preferably comprising:
a. one capsule of 400 mg of St.John's Wort;
b. one capsule of 280 mg of Echinacea;
c. one capsule of 180 to 250 mg of bee propilus; and
d. one capsule of 500 to 1000 mg of Vitamin C.

2. The items listed in (1) above to form a dengue fever kit with the appropriate dosages listed as following:
a. one capsule item (1a) every 2 to 3 hours in a 12 hour period;
b. one capsule item (1b) every 4 hours in a 12 hour period;
c. one capsule item (1c) every 4 hours in a 12 hour period; and
d. one capsule item (1d) every 1 to 2 hours in a 12 hour period.
